(19) United States
(12) Patent Application Publication
(10) Pub. No.: US 2021/0139880 A1
(43) Pub. Date: May 13, 2021

(54) Microbial Conductive Ceramics and Preparation Method and Application thereof

(71) Applicant: Jiangnan University, Wuxi (CN)

(72) Inventors: Minjie GAO, Wuxi (CN); Xiaobei ZHAN, Wuxi (CN); Zhitao LI, Wuxi (CN); Yun JIANG, Wuxi (CN); Jianrong WU, Wuxi (CN); Hongtao ZHANG, Wuxi (CN); Jiajun YAN, Wuxi (CN)

(21) Appl. No.: 17/126,346

(22) Filed: Dec. 18, 2020

Related U.S. Application Data
(63) Continuation of application No. PCT/CN2018/123379, filed on Dec. 25, 2018.

(30) Foreign Application Priority Data
Dec. 19, 2018 (CN) 201811558546.3
Dec. 20, 2018 (CN) 201811562188.3
Dec. 21, 2018 (CN) 201811569265.8

(51) Int. Cl. C12N 11/14; C04B 41/46; C04B 41/45; C04B 41/82; C04B 41/00; C12N 1/16; C12N 1/20; C12N 1/14

(52) U.S. Cl. CPC C12N 11/14; C04B 41/46; C04B 41/4539; C04B 41/82; C04B 2235/606; C12N 1/16; C12N 1/20; C12N 1/14; C04B 41/009

(57) ABSTRACT

The disclosure discloses microbial conductive ceramics and a preparation method and application thereof, and belongs to the technical field of microorganisms and the technical field of semiconductor materials. The disclosure is based on ordinary insulating macroporous ceramics, using the means of cell immobilization and the principle of microbial adsorption, to prepare the microbial conductive ceramics including macroporous ceramics, microbes immobilized on the macroporous ceramics and metal ions adsorbed to the microbes. The microbial conductive ceramics have excellent performance, and the conductivity of the microbial conductive ceramics can reach 2.91×10⁶ S/m. At the same time, the cost of the microbial conductive ceramics is low, only 10% of the cost of conductive ceramics with the same conductivity.

### TECHNICAL FIELD

The disclosure relates to microbial conductive ceramics and a preparation method and application thereof, and belongs to the technical field of microorganisms and the technical field of semiconductor materials.

### BACKGROUND

Typically, ceramics are non-conductive and thus be good insulators, for example, oxide ceramics. Since outer electrons of oxide ceramic atoms are usually bound around their respective atoms and cannot move freely under the attraction of nucleus, the oxide ceramics are usually non-conductive insulators. However, when certain oxide ceramics are heated, the outer electrons of the atom can obtain enough energy to overcome the attraction of the nucleus and become free electrons that can move freely. At this time, the oxide ceramics gain conductivity and become conductive ceramics.

At present, conductive ceramics, as a new type of semiconductor material, have been widely used in motor electrodes, electric heating elements and electronic cameras due to the advantages of oxidation resistance, high temperature resistance and metallic conductive properties, and have important application in the fields of aviation, machinery, metallurgy, electronics, etc.

However, as for the existing conductive ceramics, such as silicon nitride ceramics, zirconia ceramics, and titanium aluminum carbide ceramics, since the main oxides that constitutes the electronic conductivity are doped with impurities such as ZrO₂, ThO₂ and LaCrO₂, a heating temperature as high as 3000-5000° C. is required during preparation, and the preparation cost is relatively high. In addition, these impurities will also cause the conductivity at room temperature to be low, and the resistivity at 800° C. or above to drop, which undoubtedly greatly reduces conductive properties.

The above defects have severely restricted the progress of industrialization of conductive ceramics and their applications in aviation, machinery, metallurgy, electronics and other fields. Therefore, it is very important to find ways to reduce the cost of preparing conductive ceramics while improving their conductive properties.

### SUMMARY

The disclosure provides microbial conductive ceramics and a preparation method and application thereof. The disclosure is based on ordinary insulating macroporous ceramics, using the means of cell immobilization and the principle of microbial adsorption, to prepare the microbial conductive ceramics including macroporous ceramics, microbes immobilized on the macroporous ceramics and metal ions adsorbed to the microbes. The microbial conductive ceramics have excellent performance, and the conductivity of the microbial conductive ceramics can reach 2.91×10⁶ S/m. At the same time, the cost of the microbial conductive ceramics is low, only 10% of the cost of conductive ceramics with the same conductivity.

The disclosure provides a preparation method of microbial conductive ceramics, including: culturing microbes in a culture medium to a logarithmic growth phase or a stable phase to obtain a microbial bacterial solution; soaking the macroporous ceramics in a hydrochloric acid or sodium hydroxide solution and then drying the macroporous ceramics for the first time to obtain pretreated macroporous ceramics; placing the pretreated macroporous ceramics into the microbial bacterial solution for shaking and then drying the macroporous ceramics for the second time to obtain macroporous ceramics with immobilized microbes; and passing a metal ion solution through the macroporous ceramics with immobilized microbes, and drying the macroporous ceramics for the third time to obtain the microbial conductive ceramics, wherein the microbes include saccharomycetes, filamentous fungi or bacteria.

In one embodiment of the disclosure, the saccharomycetes include Saccharomyces cerevisiae and/or Pichia pastoris; the filamentous fungi include one or more of Aspergillus niger, Aspergillus oryzae or Mucor; and the bacteria include Escherichia coli and/or magnetotactic bacteria.

In one embodiment of the disclosure, the magnetotactic bacteria include Aquaspirillum and/or Bilophococcus.

In one embodiment of the disclosure, when the microbes are saccharomycetes, the culture time of the microbes in the culture medium is 12-60 h; when the microbes are filamentous fungi, the culture time of the microbes in the culture medium is 24-72 h; and when the microbes are bacteria, the culture time of the microbes in the culture medium is 48-96 h.

In one embodiment of the disclosure, when the microbes are saccharomycetes, the cell concentration of the microbial bacterial solution is 1×10⁶-1×10¹⁰ cells/mL; when the microbes are filamentous fungi, the cell concentration of the microbial bacterial solution is 1×10⁶-1×10⁸ cells/mL; and when the microbes are bacteria, the cell concentration of the microbial bacterial solution is 1×10⁸-1×10¹⁰ cells/mL.

In one embodiment of the disclosure, when the microbes are saccharomycetes, the cell concentration of the microbial bacterial solution is 1×10⁸ cells/mL; when the microbes are filamentous fungi, the cell concentration of the microbial bacterial solution is 1×10⁷ cells/mL; and when the microbes are bacteria, the cell concentration of the microbial bacterial solution is 1×10⁹cells/mL.

In one embodiment of the disclosure, when the microbes are saccharomycetes, filamentous fungi or bacteria, the macroporous ceramics include one or more of silicon nitride ceramics, alumina ceramics, zirconia ceramics or titanium aluminum carbide ceramics.

In one embodiment of the disclosure, when the microbes are saccharomycetes, the pore size of macroporous ceramics is 10-20 μm; when the microbes are filamentous fungi, the pore size of macroporous ceramics is 50-200 μm; and when the microbes are bacteria, the pore size of macroporous ceramics is 1-10 μm.

In one embodiment of the disclosure, when the microbes are saccharomycetes, filamentous fungi or bacteria, the concentration of the hydrochloric acid is 0.5-1.5 mol/L.

In one embodiment of the disclosure, when the microbes are saccharomycetes, filamentous fungi or bacteria, the concentration of the sodium hydroxide is 0.5-1.5 mol/L.

In one embodiment of the disclosure, when the microbes are saccharomycetes, filamentous fungi or bacteria, the soaking conditions are temperature 20-30° C., and time 24-48 h.

In one embodiment of the disclosure, when the microbes are saccharomycetes, the shaking conditions are rotation speed 50-100 r/min, temperature 30-50° C., and time 60-150 min; when the microbes are filamentous fungi, the shaking conditions are rotation speed 120-200 r/min, temperature 20-40° C., and time 4-8 h; and when the microbes are bacteria, the shaking conditions are rotation speed 20-60 r/min, temperature 40-60° C., and time 120-240 min.

In one embodiment of the disclosure, when the microbes are saccharomycetes, the shaking conditions are rotation speed 70 r/min, temperature 40° C., and time 100 min; when the microbes are filamentous fungi, the shaking conditions are rotation speed 160 r/min, temperature 30° C., and time 6 h; and when the microbes are bacteria, the shaking conditions are rotation speed 40 r/min, temperature 50° C., and time 180 min.

In one embodiment of the disclosure, when the microbes are saccharomycetes or filamentous fungi, the concentration of the metal ion solution is 30-100 mg/mL; and when the microbes are bacteria, the concentration of the metal ion solution is 50-80 mg/mL.

In one embodiment of the disclosure, when the microbes are saccharomycetes, the concentration of the metal ion solution is 50 mg/mL; and when the microbes are filamentous fungi or bacteria, the concentration of the metal ion solution is 60 mg/mL.

In one embodiment of the disclosure, when the microbes are saccharomycetes, filamentous fungi or bacteria, the pH of the metal ion solution is 2-5.

In one embodiment of the disclosure, when the microbes are saccharomycetes or filamentous fungi, the pH of the metal ion solution is 3; and when the microbes are bacteria, the pH of the metal ion solution is 4.

In one embodiment of the disclosure, when the microbes are saccharomycetes, the conditions for passing the metal ion solution through the macroporous ceramics with immobilized microbes are temperature 15-35° C., flow rate 10-30 mL/min, and time 30-120 min; when the microbes are filamentous fungi, the conditions for passing the metal ion solution through the macroporous ceramics with immobilized microbes are temperature 45-55° C., flow rate 20-40 mL/min, and time 150-240 min; and when the microbes are bacteria, the conditions for passing the metal ion solution through the macroporous ceramics with immobilized microbes are temperature 35-45° C., flow rate 5-20 mL/min, and time 60-150 min.

In one embodiment of the disclosure, when the microbes are saccharomycetes, the conditions for passing the metal ion solution through the macroporous ceramics with immobilized microbes are temperature 25° C., flow rate 20 mL/min, and time 60 min; when the microbes are filamentous fungi, the conditions for passing the metal ion solution through the macroporous ceramics with immobilized microbes are temperature 50° C., flow rate 30 mL/min, time 200 min; and when the microbes are bacteria, the conditions for passing the metal ion solution through the macroporous ceramics with immobilized microbes are temperature 40° C., flow rate 10 mL/min, and time 90 min.

The disclosure provides microbial conductive ceramics prepared by the above method.

The disclosure provides microbial conductive ceramics, including macroporous ceramics, microbes immobilized on the macroporous ceramics and metal ions adsorbed to the microbes. The microbes include saccharomycetes, filamentous fungi or bacteria.

In one embodiment of the disclosure, the saccharomycetes include S. cerevisiae and/or P. pastoris; the filamentous fungi include one or more of A. niger, A. oryzae or Mucor; and the bacteria include E. coli and/or magnetotactic bacteria.

In one embodiment of the disclosure, the magnetotactic bacteria include Aquaspirillum and/or Bilophococcus.

In one embodiment of the disclosure, when the microbes are saccharomycetes, filamentous fungi or bacteria, the macroporous ceramics include one or more of silicon nitride ceramics, alumina ceramics, zirconia ceramics or titanium aluminum carbide ceramics.

In one embodiment of the disclosure, when the microbes are saccharomycetes, the pore size of macroporous ceramics is 10-20 μm; when the microbes are filamentous fungi, the pore size of macroporous ceramics is 50-200 μm; and when the microbes are bacteria, the pore size of macroporous ceramics is 1-10 μm.

In one embodiment of the disclosure, when the microbes are saccharomycetes, the number of microbes immobilized on the macroporous ceramics is 1.0×10⁸-2.0×10⁸/cm³; when the microbes are filamentous fungi, the number of microbes immobilized on the macroporous ceramics is 1.0×10⁷-1.5×10⁷/cm³; and when the microbes are bacteria, the number of microbes immobilized on the macroporous ceramics is 1.0×10⁹-1.5×10⁹/cm³.

In one embodiment of the disclosure, when the microbes are saccharomycetes, filamentous fungi or bacteria, the metal ions include one or more of silver ion, molybdenum ion, aluminum ion, or copper ion.

In one embodiment of the disclosure, when the microbes are saccharomycetes, filamentous fungi or bacteria, the metal ion is molybdenum ion.

The disclosure provides products including the microbial conductive ceramics prepared above or the microbial conductive ceramics prepared above.

In one embodiment of the disclosure, the products include electronic components, electric heating elements, electrodes, batteries, electronic cameras, televisions, radios, computers or mobile televisions.

The disclosure provides the preparation method or the microbial conductive ceramics prepared above or application of the microbial conductive ceramics above in preparation of electronic products and measuring tool.

The microbial conductive ceramics of the disclosure has excellent performance. When the microbes are saccharomycetes, the number of the immobilized microbial cells can reach 1×10⁸ cells/cm³ or above, and the conductivity can reach 2.91×10⁶ S/m; when the microbes are filamentous fungi, the number of the immobilized microbial cells can reach 1×10⁷ cells/cm³ or above, and the conductivity can reach 2.71×10⁶ S/m; and when the microbes are bacteria, the number of the immobilized microbial cells can reach 1×10⁹/cm³ or above, and the conductivity can reach 2.51×10⁶ S/m.

To achieve the same conductivity as the disclosure, the existing conductive ceramics require ultra-high temperature sintering operation, the cost is high and the operation is complicated; while the microbial conductive ceramics of the disclosure can be prepared by only three steps of culturing microbes, attaching the microbes to macroporous ceramics, and adsorbing metal ions to microbes, the cost is low (only 10% of the cost of conductive ceramics with the same conductivity) and the operation is simple.

The microbial conductive ceramic of the disclosure has the advantages of superior performance, simple preparation and low cost, can be widely used for preparing electronic products and measuring tool, and has great application prospects.

### DETAILED DESCRIPTION

The disclosure is further described below in conjunction with specific examples.

The shaker involved in the following examples is model RH-100 purchased from Changzhou Runhua Electric Technology Co., Ltd. The S. cerevisiae involved in the following examples is S. cerevisiae CICC1221 deposited in the Microorganism Collection Center of Jiangnan University. The P. pastoris involved in the following examples is P. pastoris GS115 deposited in the Microorganism Collection Center of Jiangnan University. The A. niger involved in the following examples is A. niger CGMCC No. 14630 deposited in the Microorganism Collection Center of Jiangnan University. The A. oryzae involved in the following examples is A. oryzae CGMCC NO. 12378 deposited in the Microorganism Collection Center of Jiangnan University. The E. coli involved in the following examples is E. coli TOP10 deposited in the Microorganism Collection Center of Jiangnan University. The magnetotactic bacteria involved in the following examples is magnetotactic bacteria AMB-1 deposited in the Microorganism Collection Center of Jiangnan University. The macroporous ceramics involved in the following examples are from Dalian Institute of Chemical Physics, Chinese Academy of Sciences (The above strains A. niger CGMCC No. 14630, A. oryzae CGMCC NO. 12378, S. cerevisiae CICC122, P. pastoris GS115, E. coli TOP10, and magnetotactic bacteria AMB-1 can all be purchased without deposit for patent procedures).

The culture media involved in the disclosure are as follows:

Saccharomycetes: Seed medium: beef extract 3 g/L, peptone 10 g/L, sodium chloride 5 g/L, pH 7.4-7.6; Fermentation medium: glucose 100 g/L, peptone 20 g/L, potassium hydrogen phosphate 3 g/L, magnesium sulfate 1 g/L.

Filamentous fungi: Seed medium: potato 200 g/L, glucose 20 g/L, agar 15-20 g/L, pH natural; Fermentation medium: potato 200 g/L, glucose 20 g/L, agar 15-20 g/L, pH natural.

Bacteria: Seed medium: beef extract 3 g/L, peptone 10 g/L, sodium chloride 5 g/L, pH 7.4-7.6; Fermentation medium: beef extract 3 g/L, peptone 10 g/L, sodium chloride 5 g/L, pH 7.4-7.6.

The detection methods involved in the disclosure are as follows:

1. Calculation of dry cell weight:

The absorbance (OD₆₀₀) of a microbial bacterial solution at 600 nm is detected to obtain the cell concentration, and the dry cell weight is obtained according to the curve DCW=0.25×OD₆₀₀.

2. Determination of molybdenum ion concentration:

An Inductively Coupled Plasma-Atomic Emission Spectrometer (ICP-OES) is used, and the determination method can refer to the paper: Xie Weihua, etc.; Determination of Molybdenum Content in U-Mo Alloy by ICP-AES Method; Analysis Laboratory; 2016-04.

3. Determination of molybdenum ion adsorption:

The following formula is used for calculation: adsorption=(initial concentration-final concentration)×volume of solution/mass of adsorbent;

wherein the initial concentration is the initial concentration (mg·L⁻¹) of molybdenum ions in the molybdenum ion solution, the final concentration is the concentration (mg·L⁻¹) of molybdenum ions in the molybdenum ion solution after the molybdenum ions are adsorbed by bacterial cells, and the mass of the adsorbent is the mass corresponding to the dry weight of the adsorbent (that is, the dry cell weight).

4. Determination of the number of immobilized cells:

The microbial bacterial solution before and after being shaken together with the treated macroporous ceramics is centrifuged at 5000 r/min for 15 min respectively. The supernatant is poured out, and wet bacterial cells are obtained by centrifugation. 0.1 mL of wet bacterial cells is added into sterile water to make the volume to 100 mL, and the solution is mixed uniformly. A blood counting chamber is used for determination (for example, the average number of cells in 16 wells of the counting chamber is 4, that is, the number of cells per milliliter=4*104*25*1000=1×10⁹), and the number of original microbial cells and the number of remaining microbial cells in the microbial bacterial solution are obtained.

The following formula is used for calculation: number of immobilized cells=number of original microbial cells-number of remaining microbial cells.

5. Scanning electron microscope:

The macroporous ceramics with immobilized S. cerevisiae and the macroporous ceramics with immobilized P. pastoris are washed 3 times with deionized water and then freeze-dried. Conductive adhesive is pasted on an SEM sample stage, the sample powder is spread on the conductive adhesive, and the sample is coated with a carbon film. The sample is observed with SEM, the accelerating voltage is 15 kV, the instrument model is environmental scanning electron microscope Hitachi TM3030 (Tokyo, Japan), and thus whether the microbes are successfully attached is judged.

6. Determination of conductivity:

The conductivity of ceramics is measured using a TX-1000A intelligent metal conductor resistivity meter.

### EXAMPLE 1-1

### Effect of Pretreatment on Immobilization of Microbes on Macroporous Ceramics (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in distilled water, hydrochloric acid with concentrations of 0.5 mol/L, 1 mol/L and 1.5 mol/L, and sodium hydroxide with concentrations of 0.5 mol/L, 1 mol/L and 1.5 mol/L respectively for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 40° C. for 100 min, and dried at 105° C. for 12 h to obtain macroporous ceramics with immobilized S. cerevisiae and macroporous ceramics with immobilized P. pastoris.

The number of microbes immobilized on the macroporous ceramics was detected.

The detection results are: on the macroporous ceramics treated with distilled water, the number of immobilized S. cerevisiae cells is 2.5×10⁷ cells/cm³, and the number of immobilized P. pastoris cells is 1.1×10⁸ cells/cm³; on the macroporous ceramics treated with hydrochloric acid with a concentration of 0.5 mol/L, the number of immobilized S. cerevisiae cells is 1.3×10⁸ cells/cm³, and the number of immobilized P. pastoris cells is 1.3×10⁸ cells/cm³; on the macroporous ceramics treated with hydrochloric acid with a concentration of 1 mol/L, the number of immobilized S. cerevisiae cells is 1.6×10⁸ cells/cm³, and the number of immobilized P. pastoris cells is 1.5×10⁸ cells/cm³; on the macroporous ceramics treated with hydrochloric acid with a concentration of 1.5 mol/L, the number of immobilized S. cerevisiae cells is 1.1×10⁸ cells/cm³, and the number of immobilized P. pastoris cells is 1.6×10⁸ cells/cm³; on the macroporous ceramics treated with sodium hydroxide with a concentration of 0.5 mol/L, the number of immobilized S. cerevisiae cells is 1.2×10⁸ cells/cm³, and the number of immobilized P. pastoris cells is 1.3×10⁸ cells/cm³; on the macroporous ceramics treated with sodium hydroxide with a concentration of 1 mol/L, the number of immobilized S. cerevisiae cells is 1.5×10⁸ cells/cm³, and the number of immobilized P. pastoris cells is 1.4×10⁸ cells/cm³; and on the macroporous ceramics treated with sodium hydroxide with a concentration of 1.5 mol/L, the number of immobilized S. cerevisiae cells is 1.4×10⁸ cells/cm³, and the number of immobilized P. pastoris cells is 1.6×10⁸ cells/cm³.

Therefore, 1 mol hydrochloric acid or 1.5 mol sodium hydroxide shall be used to treat the macroporous ceramics to make the ceramics carry more positive charges or negative charges. Under such condition, the saccharomycetes can better adhere to the gaps inside the ceramics by electrostatic adsorption, so that the inside of the ceramics is better filled, the metal adsorption rate is improved, and further the conductivity is higher.

### EXAMPLE 1-2

### Effect of Temperature on Immobilization of Microbes on Macroporous Ceramics (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 20° C., 30° C., 40° C. and 50° C. for 100 min, and dried at 105° C. for 12 h to obtain macroporous ceramics with immobilized S. cerevisiae and macroporous ceramics with immobilized P. pastoris.

The number of microbes immobilized on the macroporous ceramics was detected.

The detection results are: on the macroporous ceramics treated at 20° C., the number of immobilized S. cerevisiae cells is 1.2×10⁸ cells/cm³, and the number of immobilized P. pastoris cells is 1.3×10⁸ cells/cm³; on the macroporous ceramics treated at 30° C., the number of immobilized S. cerevisiae cells is 1.3×10⁸ cells/cm³, and the number of immobilized P. pastoris cells is 1.4×10⁸ cells/cm³; on the macroporous ceramics treated at 40° C., the number of immobilized S. cerevisiae cells is 1.6×10⁸ cells/cm³, and the number of immobilized P. pastoris cells is 1.6×10⁸ cells/cm³; and on the macroporous ceramics treated at 50° C., the number of immobilized S. cerevisiae cells is 1.4×10⁸ cells/cm³, and the number of immobilized P. pastoris cells is 1.5×10⁸ cells/cm³.

Therefore, the macroporous ceramics shall be treated at 40° C. to make the saccharomycetes better adhere to the gaps inside the ceramics, so that the inside of the ceramics is better filled, the metal adsorption rate is improved, and further the conductivity is higher.

### EXAMPLE 1-3

### Effect of Rotation Speed on Immobilization of Microbes on Macroporous Ceramics (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 50 r·min⁻¹, 60 r·min⁻¹, 70 r·min⁻¹, 80 r·min⁻¹, 90 r·min⁻¹ and 100 r·min⁻¹ and 40° C. for 100 min, and dried at 105° C. for 12 h to obtain macroporous ceramics with immobilized S. cerevisiae and macroporous ceramics with immobilized P. pastoris.

The number of microbes immobilized on the macroporous ceramics was detected.

The detection results are: on the macroporous ceramics treated at 50 r·min⁻¹, the number of immobilized S. cerevisiae cells is 1.2×10⁸ cells/cm³, and the number of immobilized P. pastoris cells is 1.3×10⁸ cells/cm³; on the macroporous ceramics treated at 60 r·min⁻¹, the number of immobilized S. cerevisiae cells is 1.4×10⁸ cells/cm³, and the number of immobilized P. pastoris cells is 1.4×10⁸ cells/cm³; on the macroporous ceramics treated at 70 r·min⁻¹, the number of immobilized S. cerevisiae cells is 1.4×10⁸ cells/cm³, and the number of immobilized P. pastoris cells is 1.7×10⁸ cells/cm³; on the macroporous ceramics treated at 80 r·min⁻¹, the number of immobilized S. cerevisiae cells is 1.4×10⁸ cells/cm³, and the number of immobilized P. pastoris cells is 1.5×10⁸ cells/cm³; on the macroporous ceramics treated at 90 r·min⁻¹, the number of immobilized S. cerevisiae cells is 1.4×10⁸ cells/cm³, and the number of immobilized P. pastoris cells is 1.5×10⁸ cells/cm³; and on the macroporous ceramics treated at 100 r·min⁻¹, the number of immobilized S. cerevisiae cells is 1.3×10⁸ cells/cm³, and the number of immobilized P. pastoris cells is 1.5×10⁸ cells/cm³.

Therefore, the macroporous ceramics shall be treated at 70 r·min⁻¹ to make the saccharomycetes better adhere to the gaps inside the ceramics without being thrown off, so that the inside of the ceramics is better filled, the metal adsorption rate is improved, and further the conductivity is higher.

### EXAMPLE 1-4

### Effect of Flow Rate on Adsorption of Metal Ions by Microbes (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 40° C. for 100 min, and dried at 105° C. for 12 h to obtain macroporous ceramics with immobilized S. cerevisiae and macroporous ceramics with immobilized P. pastoris.

(7) The macroporous ceramics with immobilized S. cerevisiae and the macroporous ceramics with immobilized P. pastoris were respectively fixed in soft catheters with two ends mutually communicated, and the catheters were connected to a peristaltic pump. The flow rate of the peristaltic pump was adjusted to 10 mL/min, 15 mL/min, 20 mL/min, 25 mL/min and 30 mL/min, and the two ends of the catheter were put into an ion solution with a molybdenum ion concentration of 50 mg/mL and a pH of 3. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 2 h to obtain microbial conductive ceramics.

The amount of metal ions adsorbed by microbes is detected.

The detection results are: on the macroporous ceramics treated at a flow rate of 10 mL/min, the amount of metal ions adsorbed by S. cerevisiae is 1.1 mmol/g, and the amount of metal ions adsorbed by P. pastoris is 1.2 mmol/g; on the macroporous ceramics treated at a flow rate of 15 mL/min, the amount of metal ions adsorbed by S. cerevisiae is 1.3 mmol/g, and the amount of metal ions adsorbed by P. pastoris is 1.5 mmol/g; on the macroporous ceramics treated at a flow rate of 20 mL/min, the amount of metal ions adsorbed by S. cerevisiae is 1.6 mmol/g, and the amount of metal ions adsorbed by P. pastoris is 1.5 mmol/g; on the macroporous ceramics treated at a flow rate of 25 mL/min, the amount of metal ions adsorbed by S. cerevisiae is 1.5 mmol/g, and the amount of metal ions adsorbed by P. pastoris is 1.5 mmol/g; and on the macroporous ceramics treated at a flow rate of 30 mL/min, the amount of metal ions adsorbed by S. cerevisiae is 1.4 mmol/g, and the amount of metal ions adsorbed by P. pastoris is 1.5 mmol/g.

Therefore, the macroporous ceramics shall be treated at a flow rate of 20 mL/min.

### EXAMPLE 1-5

### Effect of pH on Adsorption of Metal Ions by Microbes (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 40° C. for 100 min, and dried at 105° C. for 12 h to obtain macroporous ceramics with immobilized S. cerevisiae and macroporous ceramics with immobilized P. pastoris.

(7) The macroporous ceramics with immobilized S. cerevisiae and the macroporous ceramics with immobilized P. pastoris were respectively fixed in soft catheters with two ends mutually communicated, and the catheters were connected to a peristaltic pump. The flow rate of the peristaltic pump was adjusted to 20 mL/min, and the two ends of the catheter were put into ion solutions with a molybdenum ion concentration of 50 mg/mL and a pH of 1, 2, 3, 4 and 5. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 12 h to obtain microbial conductive ceramics.

The amount of metal ions adsorbed by microbes is detected.

The detection results are: on the macroporous ceramics treated at a pH of 1, the amount of metal ions adsorbed by S. cerevisiae is 1.0 mmol/g, and the amount of metal ions adsorbed by P. pastoris is 1.3 mmol/g; on the macroporous ceramics treated at a pH of 2, the amount of metal ions adsorbed by S. cerevisiae is 1.3 mmol/g, and the amount of metal ions adsorbed by P. pastoris is 1.4 mmol/g; on the macroporous ceramics treated at a pH of 3, the amount of metal ions adsorbed by S. cerevisiae is 1.5 mmol/g, and the amount of metal ions adsorbed by P. pastoris is 1.6 mmol/g; on the macroporous ceramics treated at a pH of 4, the amount of metal ions adsorbed by S. cerevisiae is 1.4 mmol/g, and the amount of metal ions adsorbed by P. pastoris is 1.5 mmol/g; and on the macroporous ceramics treated at a pH of 5, the amount of metal ions adsorbed by S. cerevisiae is 1.4 mmol/g, and the amount of metal ions adsorbed by P. pastoris is 1.5 mmol/g.

Therefore, the macroporous ceramics shall be treated at a pH of 3.

### EXAMPLE 1-6

### Effect of Microbial Culture Time on Adsorption of Metal Ions by Microbes (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 12 h, 24 h, 36 h, 48 h and 60 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 40° C. for 100 min, and dried at 105° C. for 12 h to obtain macroporous ceramics with immobilized S. cerevisiae and macroporous ceramics with immobilized P. pastoris.

(7) The macroporous ceramics with immobilized S. cerevisiae and the macroporous ceramics with immobilized P. pastoris were respectively fixed in soft catheters with two ends mutually communicated, and the catheters were connected to a peristaltic pump. The flow rate of the peristaltic pump was adjusted to 70 mL/min, and the two ends of the catheter were put into an ion solution with a molybdenum ion concentration of 50 mg/mL and a pH of 3. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 2 h to obtain microbial conductive ceramics.

The amount of metal ions adsorbed by microbes is detected.

The detection results are: after fermentation culture for 12 h, the amount of metal ions adsorbed by S. cerevisiae is 1.1 mmol/g, and the amount of metal ions adsorbed by P. pastoris is 1.2 mmol/g; after fermentation culture for 24 h, the amount of metal ions adsorbed by S. cerevisiae is 1.4 mmol/g, and the amount of metal ions adsorbed by P. pastoris is 1.5 mmol/g; after fermentation culture for 36 h, the amount of metal ions adsorbed by S. cerevisiae is 1.6 mmol/g, and the amount of metal ions adsorbed by P. pastoris is 1.6 mmol/g; after fermentation culture for 48 h, the amount of metal ions adsorbed by S. cerevisiae is 1.5 mmol/g, and the amount of metal ions adsorbed by P. pastoris is 1.6 mmol/g; and after fermentation culture for 60 h, the amount of metal ions adsorbed by S. cerevisiae is 1.6 mmol/g, and the amount of metal ions adsorbed by P. pastoris is 1.6 mmol/g.

Therefore, treatment with the microbial macroporous ceramics subjected to fermentation culture for 12-60 h has good effects. It may be because the saccharomycetes is in the logarithmic growth phase, the stable phase or the transition phase from the logarithmic growth phase to the stable phase, the cell membrane has better permeability, and the metal ions can be absorbed more easily.

### EXAMPLE 1-7

### Preparation of Microbial Conductive Ceramics (Saccharomycetes)

Specific steps are as follows:

(1) A single colony of S. cerevisiae was picked from a plate, inoculated into a 500 mL Erlenmeyer flask pre-added with 50 mL of seed medium, and cultured for 24 h in a shaker at 30° C. and 220 r·min⁻¹ to obtain a S. cerevisiae seed solution.

(2) The S. cerevisiae seed solution was inoculated into a 5 L fermenter pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution was centrifuged at 1500 r·min⁻¹ for 15 min to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells.

(4) The S. cerevisiae cells were put into distilled water, and the cell concentration was controlled at 1×10⁸ cells/mL to obtain a S. cerevisiae bacterial solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution, shaken on a shaker at 70 r·min⁻¹ and 40° C. for 100 min, and dried at 105° C. for 12 h to obtain macroporous ceramics with immobilized S. cerevisiae.

(7) The macroporous ceramics with immobilized S. cerevisiae were fixed in a soft catheter with two ends mutually communicated, and the catheter was connected to a peristaltic pump. The flow rate of the peristaltic pump was adjusted to 20 mL/min, and the two ends of the catheter were put into an ion solution with a molybdenum ion concentration of 50 mg/mL and a pH of 3. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 2 h to obtain microbial conductive ceramics. The conductive properties of the microbial conductive ceramics were detected, and the conductivity was 2.85×10⁶ S/m.

### EXAMPLE 1-8

### Preparation of Microbial Conductive Ceramics (Saccharomycetes)

Specific steps are as follows:

(1) A single colony of P. pastoris was picked from a plate, inoculated into a 500 mL Erlenmeyer flask pre-added with 50 mL of seed medium, and cultured for 24 h in a shaker at 30° C. and 220 r·min⁻¹ to obtain a P. pastoris seed solution.

(2) The P. pastoris seed solution was inoculated into a 5 L fermenter pre-added with 1.2 L of fermentation medium at an inoculum amount of 10% (that is, the volume of the seed solution accounted for 10% of the volume of the fermentation medium), and fermented at 37° C. for 36 h to obtain a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained P. pastoris fermentation solution was centrifuged at 1500 r·min⁻1 for 15 min to obtain bacterial cells; the bacterial cells were washed with distilled water and centrifuged at 1500 r·min⁻¹ for 5 min, and live bacterial cells were collected; and the washing operation was repeated 3 times to obtain P. pastoris cells.

(4) The P. pastoris cells were put into distilled water, and the cell concentration was controlled at 1×10⁸ cells/mL to obtain a P. pastoris bacterial solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the P. pastoris solution, shaken on a shaker at 70 r·min⁻¹ and 40° C. for 100 min, and dried at 105° C. for 12 h to obtain macroporous ceramics with immobilized P. pastoris.

(7) The macroporous ceramics with immobilized P. pastoris were fixed in a soft catheter with two ends mutually communicated, and the catheter was connected to a peristaltic pump. The flow rate of the peristaltic pump was adjusted to 20 mL/min, and the two ends of the catheter were put into an ion solution with a molybdenum ion concentration of 50 mg/mL and a pH of 3. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 2 h to obtain microbial conductive ceramics. The conductive properties of the microbial conductive ceramics were detected, and the conductivity was 2.87×10⁶ S/m.

### EXAMPLE 1-9

### Preparation of Microbial Conductive Ceramics (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 40° C. for 100 min, and dried at 105° C. for 12 h to obtain macroporous ceramics with immobilized S. cerevisiae and macroporous ceramics with immobilized P. pastoris.

(7) The macroporous ceramics with immobilized S. cerevisiae and the macroporous ceramics with immobilized P. pastoris were respectively fixed in soft catheters with two ends mutually communicated, and the catheters were connected to a peristaltic pump. The flow rate of the peristaltic pump was adjusted to 20 mL/min, and the two ends of the catheter were put into an ion solution with a molybdenum ion concentration of 50 mg/mL and a pH of 3. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 2 h to obtain microbial conductive ceramics. The conductive properties of the microbial conductive ceramics were detected, and the conductivity was 2.86×10⁶ S/m.

### EXAMPLE 1-10

### Preparation of Microbial Conductive Ceramics (Saccharomycetes)

Specific steps are as follows:

(1) A single colony of S. cerevisiae and a single colony of P. pastoris were picked from plates, inoculated into 500 mL Erlenmeyer flasks pre-added with 50 mL of seed medium respectively, and cultured in shakers at 30° C. and 220 r·min⁻¹ for 24 h to obtain a S. cerevisiae seed solution and a P. pastoris seed solution.

(2) The S. cerevisiae seed solution and the P. pastoris seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 36 h to obtain a S. cerevisiae fermentation solution and a P. pastoris fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained S. cerevisiae fermentation solution and P. pastoris fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain S. cerevisiae cells and P. pastoris cells.

(4) The S. cerevisiae cells and the P. pastoris cells were put into distilled water respectively, and the cell concentration was controlled at 1×10⁸ cells/mL to obtain a S. cerevisiae solution and a P. pastoris solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the S. cerevisiae solution and the P. pastoris solution respectively, shaken on a shaker at 70 r·min⁻¹ and 40° C. for 100 min, and dried at 105° C. for 12 h to obtain macroporous ceramics with immobilized S. cerevisiae and macroporous ceramics with immobilized P. pastoris.

(7) The macroporous ceramics with immobilized S. cerevisiae and the macroporous ceramics with immobilized P. pastoris were respectively fixed in soft catheters with two ends mutually communicated, and the catheters were connected to a peristaltic pump.

The flow rate of the peristaltic pump was adjusted to 20 mL/min, and the two ends of the catheter were put into ion solutions with a concentration of 50 mg/mL of silver ions, copper ions and aluminum ions and a pH of 3 respectively. The peristaltic pump was turned on at 25° C., and the metal ion concentrate was fed to the soft catheter. The concentrate slowly passed through the ceramics, and then the metal ions were adsorbed for 60 min. After the adsorption, the macroporous ceramics with immobilized microbes were dried at 150° C. for 2 h to obtain microbial conductive ceramics. The conductive properties of the microbial conductive ceramics were detected, the experiment was repeated three times, and the conductivity was 2.91×10⁶ S/m, 2.51×10⁶ S/m and 2.46×10⁶ S/m respectively.

### EXAMPLE 2-1

### Effect of Pretreatment on Immobilization of Microbes on Macroporous Ceramics (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in distilled water, hydrochloric acid with concentrations of 0.5 mol/L, 1 mol/L and 1.5 mol/L, and sodium hydroxide with concentrations of 0.5 mol/L, 1 mol/L and 1.5 mol/L respectively for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 30° C. for 6 h, and dried at 105° C. for 12 h to obtain macroporous ceramics with immobilized A. niger and macroporous ceramics with immobilized A. oryzae.

The number of microbes immobilized on the macroporous ceramics was detected.

The detection results are: on the macroporous ceramics treated with distilled water, the number of immobilized A. niger cells is 1.1×10⁷ cells/cm³, and the number of immobilized A. oryzae cells is 1.8×10⁶ cells/cm³; on the macroporous ceramics treated with hydrochloric acid with a concentration of 0.5 mol/L, the number of immobilized A. niger cells is 1.1×10⁷ cells/cm³, and the number of immobilized A. oryzae cells is 1.1×10⁷ cells/cm³; on the macroporous ceramics treated with hydrochloric acid with a concentration of 1 mol/L, the number of immobilized A. niger cells is 1.2×10⁷ cells/cm³, and the number of immobilized A. oryzae cells is 1.3×10⁷ cells/cm³; on the macroporous ceramics treated with hydrochloric acid with a concentration of 1.5 mol/L, the number of immobilized A. niger cells is 1.1×10⁷ cells/cm³, and the number of immobilized A. oryzae cells is 1.3×10⁷ cells/cm³; on the macroporous ceramics treated with sodium hydroxide with a concentration of 0.5 mol/L, the number of immobilized A. niger cells is 1.1×10⁷ cells/cm³, and the number of immobilized A. oryzae cells is 1.2×10⁷ cells/cm³; on the macroporous ceramics treated with sodium hydroxide with a concentration of 1 mol/L, the number of immobilized A. niger cells is 1.3×10⁷ cells/cm³, and the number of immobilized A. oryzae cells is 1.3×10⁷ cells/cm³; and on the macroporous ceramics treated with sodium hydroxide with a concentration of 1.5 mol/L, the number of immobilized A. niger cells is 1.3×10⁷ cells/cm³, and the number of immobilized A. oryzae cells is 1.2×10⁷ cells/cm³.

Therefore, sodium hydroxide with a concentration of 1 mol/L shall be used to treat the macroporous ceramics to make the ceramics carry more positive charges or negative charges. Under such condition, the filamentous fungi can better adhere to the gaps inside the ceramics by electrostatic adsorption, so that the inside of the ceramics is better filled, the metal adsorption rate is improved, and further the conductivity is higher.

### EXAMPLE 2-2

### Effect of Temperature on Immobilization of Microbes on Macroporous Ceramics (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 20° C., 30° C., 40° C. and 50° C. for 6 h, and dried at 105° C. for 12 h to obtain macroporous ceramics with immobilized A. niger and macroporous ceramics with immobilized A. oryzae.

The number of microbes immobilized on the macroporous ceramics was detected.

The detection results are: on the macroporous ceramics treated at 10° C., the number of immobilized A. niger cells is 1.1×10⁷ cells/cm³, and the number of immobilized A. oryzae cells is 1.1×10⁷ cells/cm³; on the macroporous ceramics treated at 20° C., the number of immobilized A. niger cells is 1.2×10⁷ cells/cm³, and the number of immobilized A. oryzae cells is 1.3×10⁷ cells/cm³; on the macroporous ceramics treated at 30° C., the number of immobilized A. niger cells is 1.4×10⁷ cells/cm³, and the number of immobilized A. oryzae cells is 1.3×10⁷ cells/cm³; on the macroporous ceramics treated at 40° C., the number of immobilized A. niger cells is 1.3×10⁷ cells/cm³, and the number of immobilized A. oryzae cells is 1.2×10⁷ cells/cm³; and on the macroporous ceramics treated at 50° C., the number of immobilized A. niger cells is 1.3×10⁷ cells/cm³, and the number of immobilized A. oryzae cells is 1.3×10⁷ cells/cm³.

Therefore, the macroporous ceramics shall be treated at 30° C. to make the filamentous fungi better adhere to the gaps inside the ceramics, so that the inside of the ceramics is better filled, the metal adsorption rate is improved, and further the conductivity is higher.

### EXAMPLE 2-3

### Effect of Rotation Speed on Immobilization of Microbes on Macroporous Ceramics (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 120 r·min⁻¹, 140 r·min⁻¹, 160 r·min⁻¹, 180 r·min⁻¹ and 200 r·min⁻¹ and 30° C. for 6 h, and dried at 105° C. for 12 h to obtain macroporous ceramics with immobilized A. niger and macroporous ceramics with immobilized A. oryzae.

The number of microbes immobilized on the macroporous ceramics was detected.

The detection results are: on the macroporous ceramics treated at 120 r·min⁻¹, the number of immobilized A. niger cells is 1.0×10⁷ cells/cm³, and the number of immobilized A. oryzae cells is 1.2×10⁷ cells/cm³; on the macroporous ceramics treated at 140 r·min⁻¹, the number of immobilized A. niger cells is 1.2×10⁷ cells/cm³, and the number of immobilized A. oryzae cells is 1.3×10⁷ cells/cm³; on the macroporous ceramics treated at 160 r·min⁻¹, the number of immobilized A. niger cells is 1.3×10⁷ cells/cm³, and the number of immobilized A. oryzae cells is 1.4×10⁷ cells/cm³; on the macroporous ceramics treated at 180 r·min⁻¹, the number of immobilized A. niger cells is 1.3×10⁷ cells/cm³, and the number of immobilized A. oryzae cells is 1.3×10⁷ cells/cm³; and on the macroporous ceramics treated at 200 r·min⁻¹, the number of immobilized A. niger cells is 1.2×10⁷ cells/cm³, and the number of immobilized A. oryzae cells is 1.3×10⁷ cells/cm³.

Therefore, the macroporous ceramics shall be treated at 160 r·min⁻¹ to make the filamentous fungi better adhere to the gaps inside the ceramics without being thrown off, so that the inside of the ceramics is better filled, the metal adsorption rate is improved, and further the conductivity is higher.

### EXAMPLE 2-4

### Effect of Flow Rate on Adsorption of Metal Ions by Microbes (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 30° C. for 6 h, and dried at 105° C. for 12 h to obtain macroporous ceramics with immobilized A. niger and macroporous ceramics with immobilized A. oryzae.

(7) The macroporous ceramics with immobilized A. niger and the macroporous ceramics with immobilized A. oryzae were respectively fixed in soft catheters with two ends mutually communicated, and the catheters were connected to a peristaltic pump. The flow rate of the peristaltic pump was adjusted to 10 mL/min, 20 mL/min, 30 mL/min, 40 mL/min and 50 mL/min, and the two ends of the catheter were put into an ion solution with a molybdenum ion concentration of 60 mg/mL and a pH of 3. The peristaltic pump was turned on at 50° C., and the metal ion concentrate was fed to the soft catheter. The concentrate slowly passed through the ceramics, and then the metal ions were adsorbed for 200 min. After the adsorption, the macroporous ceramics with immobilized microbes were dried at 150° C. for 2 h to obtain microbial conductive ceramics.

The amount of metal ions adsorbed by microbes is detected.

The detection results are: on the macroporous ceramics treated at a flow rate of 10 mL/min, the amount of metal ions adsorbed by A. niger is 1.1 mmol/g, and the amount of metal ions adsorbed by A. oryzae 1.2 mmol/g; on the macroporous ceramics treated at a flow rate of 20 mL/min, the amount of metal ions adsorbed by A. niger is 1.2 mmol/g, and the amount of metal ions adsorbed by A. oryzae 1.3 mmol/g; on the macroporous ceramics treated at a flow rate of 30 mL/min, the amount of metal ions adsorbed by A. niger is 1.3 mmol/g, and the amount of metal ions adsorbed by A. oryzae 1.4 mmol/g; on the macroporous ceramics treated at a flow rate of 40 mL/min, the amount of metal ions adsorbed by A. niger is 1.3 mmol/g, and the amount of metal ions adsorbed by A. oryzae 1.3 mmol/g; and on the macroporous ceramics treated at a flow rate of 50 mL/min, the amount of metal ions adsorbed by A. niger is 1.4 mmol/g, and the amount of metal ions adsorbed by A. oryzae 1.2 mmol/g.

Therefore, the macroporous ceramics shall be treated at a flow rate of 30 mL/min.

### EXAMPLE 2-5

### Effect of pH on Adsorption of Metal Ions by Microbes (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 30° C. for 6 h, and dried at 105° C. for 12 h to obtain macroporous ceramics with immobilized A. niger and macroporous ceramics with immobilized A. oryzae.

(7) The macroporous ceramics with immobilized A. niger and the macroporous ceramics with immobilized A. oryzae were respectively fixed in soft catheters with two ends mutually communicated, and the catheters were connected to a peristaltic pump. The flow rate of the peristaltic pump was adjusted to 160 mL/min, and the two ends of the catheter were put into ion solutions with a molybdenum ion concentration of 60 mg/mL and a pH of 1, 2, 3, 4 and 5 respectively. The peristaltic pump was turned on at 50° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics.

The amount of metal ions adsorbed by microbes is detected.

The detection results are: on the macroporous ceramics treated at a pH of 1, the amount of metal ions adsorbed by A. niger is 1.0 mmol/g, and the amount of metal ions adsorbed by A. oryzae is 1.2 mmol/g; on the macroporous ceramics treated at a pH of 2, the amount of metal ions adsorbed by A. niger is 1.1 mmol/g, and the amount of metal ions adsorbed by A. oryzae is 1.3 mmol/g; on the macroporous ceramics treated at a pH of 3, the amount of metal ions adsorbed by A. niger is 1.3 mmol/g, and the amount of metal ions adsorbed by A. oryzae is 1.4 mmol/g; on the macroporous ceramics treated at a pH of 4, the amount of metal ions adsorbed by A. niger is 1.2 mmol/g, and the amount of metal ions adsorbed by A. oryzae is 1.3 mmol/g; and on the macroporous ceramics treated at a pH of 5, the amount of metal ions adsorbed by A. niger is 1.3 mmol/g, and the amount of metal ions adsorbed by A. oryzae is 1.3 mmol/g.

Therefore, the macroporous ceramics shall be treated at a pH of 3.

### EXAMPLE 2-6

### Effect of Time on Adsorption of Metal Ions by Microbes (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 24 h, 36 h, 48 h, 60 h and 72 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 30° C. for 6 h, and dried at 105° C. for 12 h to obtain macroporous ceramics with immobilized A. niger and macroporous ceramics with immobilized A. oryzae.

(7) The macroporous ceramics with immobilized A. niger and the macroporous ceramics with immobilized A. oryzae were respectively fixed in soft catheters with two ends mutually communicated, and the catheters were connected to a peristaltic pump. The flow rate of the peristaltic pump was adjusted to 30 mL/min, and the two ends of the catheter were put into an ion solution with a molybdenum ion concentration of 60 mg/mL and a pH of 3. The peristaltic pump was turned on at 50° C., and the metal ion concentrate was fed to the soft catheter. The concentrate slowly passed through the ceramics, and then the metal ions were adsorbed for 200 min. After the adsorption, the macroporous ceramics with immobilized microbes were dried at 150° C. for 2 h to obtain microbial conductive ceramics.

The amount of metal ions adsorbed by microbes is detected.

The detection results are: after fermentation culture for 24 h, the amount of metal ions adsorbed by A. niger is 1.0 mmol/g, and the amount of metal ions adsorbed by A. oryzae is 1.1 mmol/g; after fermentation culture for 36 h, the amount of metal ions adsorbed by A. niger is 1.2 mmol/g, and the amount of metal ions adsorbed by A. oryzae is 1.2 mmol/g; after fermentation culture for 48 h, the amount of metal ions adsorbed by A. niger is 1.3 mmol/g, and the amount of metal ions adsorbed by A. oryzae is 1.4 mmol/g; after fermentation culture for 60 h, the amount of metal ions adsorbed by A. niger is 1.3 mmol/g, and the amount of metal ions adsorbed by A. oryzae is 1.4 mmol/g; and after fermentation culture for 72 h, the amount of metal ions adsorbed by A. niger is 1.3 mmol/g, and the amount of metal ions adsorbed by A. oryzae is 1.3 mmol/g.

Therefore, treatment with the microbial macroporous ceramics subjected to fermentation culture for 24-72 h has good effects. It may be because the filamentous fungi are in the logarithmic growth phase, the stable phase or the transition phase from the logarithmic growth phase to the stable phase, the cell membrane has better permeability, and the metal ions can be absorbed more easily.

### EXAMPLE 2-7

### Preparation of Microbial Conductive Ceramics (Filamentous Fungi)

Specific steps are as follows:

(1) A single colony of A. niger was picked from a plate, inoculated into a 500 mL Erlenmeyer flask pre-added with 50 mL of seed culture medium, and cultured in a shaker at 37° C. and 220 r·min⁻¹ for 72 h to obtain an A. niger seed solution.

(2) The A. niger seed solution was inoculated into a 5 L fermenter pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution was centrifuged at 1500 r·min⁻¹ for 15 min to obtain bacterial cells; the bacterial cells were washed with distilled water and centrifuged at 1500 r·min⁻¹ for 5 min, and live bacterial cells were collected; and the washing operation was repeated 3 times to obtain A. niger cells.

(4) The A. niger cells were put into distilled water, and the cell concentration was controlled at 1×10⁷ cells/mL to obtain an A. niger solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution, shaken on a shaker at 160 r·min⁻¹ and 30° C. for 6 h, and dried at 105° C. for 12 h to obtain macroporous ceramics with immobilized A. niger.

(7) The macroporous ceramics with immobilized A. niger were fixed in a soft catheter with two ends mutually communicated, and the catheter was connected to a peristaltic pump. The flow rate of the peristaltic pump was adjusted to 160 mL/min, and the two ends of the catheter were put into an ion solution with a molybdenum ion concentration of 60 mg/mL and a pH of 3. The peristaltic pump was turned on at 50° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics. The conductive properties of the microbial conductive ceramics were detected, and the conductivity was 2.61×10⁶ S/m.

### EXAMPLE 2-8

### Preparation of Microbial Conductive Ceramics (Filamentous Fungi)

Specific steps are as follows:

(1) A single colony of A. oryzae was picked from a plate, inoculated into a 500 mL Erlenmeyer flask pre-added with 50 mL of seed culture medium, and cultured in a shaker at 37° C. and 220 r·min⁻¹ for 72 h to obtain an A. oryzae seed solution.

(2) The A. oryzae seed solution was inoculated into a 5 L fermenter pre-added with 1.2 L of fermentation medium at an inoculum amount of 10% (that is, the volume of the seed solution accounted for 10% of the volume of the fermentation medium), and fermented at 37° C. for 48 h to obtain an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. oryzae fermentation solution was centrifuged at 1500 r·min⁻¹ for 15 min to obtain bacterial cells; the bacterial cells were washed with distilled water and centrifuged at 1500 r·min⁻¹ for 5 min, and live bacterial cells were collected; and the washing operation was repeated 3 times to obtain A. oryzae cells.

(4) The A. oryzae cells were put into distilled water, and the cell concentration was controlled at 1×10⁷ cells/mL to obtain an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. oryzae solution, shaken on a shaker at 160 r·min⁻¹ and 30° C. for 6 h, and dried at 105° C. for 12 h to obtain macroporous ceramics with immobilized A. oryzae.

(7) The macroporous ceramics with immobilized A. oryzae were fixed in a soft catheter with two ends mutually communicated, and the catheter was connected to a peristaltic pump. The flow rate of the peristaltic pump was adjusted to 160 mL/min, and the two ends of the catheter were put into an ion solution with a molybdenum ion concentration of 60 mg/mL and a pH of 3. The peristaltic pump was turned on at 50° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics. The conductive properties of the microbial conductive ceramics were detected, and the conductivity was 2.63×10⁶ S/m.

### EXAMPLE 2-9

### Preparation of Microbial Conductive Ceramics (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 30° C. for 6 h, and dried at 105° C. for 12 h to obtain macroporous ceramics with immobilized A. niger and macroporous ceramics with immobilized A. oryzae.

(7) The macroporous ceramics with immobilized A. niger and the macroporous ceramics with immobilized A. oryzae were respectively fixed in soft catheters with two ends mutually communicated, and the catheters were connected to a peristaltic pump. The flow rate of the peristaltic pump was adjusted to 160 mL/min, and the two ends of the catheter were put into an ion solution with a molybdenum ion concentration of 60 mg/mL and a pH of 3. The peristaltic pump was turned on at 50° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics. The conductive properties of the microbial conductive ceramics were detected, and the conductivity was 2.65×10⁶ S/m.

### EXAMPLE 2-10

### Preparation of Microbial Conductive Ceramics (Filamentous Fungi)

Specific steps are as follows:

(1) A single colony of A. niger and a single colony of A. oryzae were picked from plates, inoculated into 500 mL Erlenmeyer flasks pre-added with 50 mL of seed medium respectively, and cultured in shakers at 37° C. and 220 r·min⁻¹ for 72 h to obtain an A. niger seed solution and an A. oryzae seed solution.

(2) The A. niger seed solution and the A. oryzae seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h to obtain an A. niger fermentation solution and an A. oryzae fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained A. niger fermentation solution and A. oryzae fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain A. niger cells and A. oryzae cells.

(4) The A. niger cells and the A. oryzae cells were put into distilled water respectively, and the cell concentration was controlled at 1×10⁷ cells/mL to obtain an A. niger solution and an A. oryzae solution.

(5) Macroporous ceramics were soaked in sodium hydroxide with a concentration of 1 mol/L for 24 h, and then dried at 105° C. for 12 h to obtain treated macroporous ceramics.

(6) The treated macroporous ceramics were put into the A. niger solution and the A. oryzae solution respectively, shaken on a shaker at 160 r·min⁻¹ and 30° C. for 6 h, and dried at 105° C. for 12 h to obtain macroporous ceramics with immobilized A. niger and macroporous ceramics with immobilized A. oryzae.

(7) The macroporous ceramics with immobilized A. niger and the macroporous ceramics with immobilized A. oryzae were respectively fixed in soft catheters with two ends mutually communicated, and the catheters were connected to a peristaltic pump. The flow rate of the peristaltic pump was adjusted to 160 mL/min, and the two ends of the catheter were put into ion solutions with a concentration of 60 mg/mL of silver ions, copper ions and aluminum ions respectively and with a pH of 3. The peristaltic pump was turned on at 50° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics. The conductive properties of the microbial conductive ceramics were detected, the experiment was repeated three times, and the conductivity was 2.71×10⁶ S/m, 2.41×10⁶ S/m and 2.35×10⁶ S/m respectively.

### EXAMPLE 3-1

### Effect of Pretreatment on Immobilization of Microbes on Macroporous Ceramics (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in distilled water, hydrochloric acid with concentrations of 0.5 mol/L, 1 mol/L and 1.5 mol/L, and sodium hydroxide with concentrations of 0.5 mol/L, 1 mol/L and 1.5 mol/L respectively for 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 50° C. for 180 min, and dried at 105° C. for 12 h to obtain macroporous ceramics with immobilized E. coli and macroporous ceramics with immobilized magnetotactic bacteria.

The number of microbes immobilized on the macroporous ceramics was detected.

The detection results are: on the macroporous ceramics treated with distilled water, the number of immobilized E. coli cells is 1.1×10⁹ cells/cm³, and the number of immobilized magnetotactic bacterium cells is 1.9×10⁸ cells/cm³; on the macroporous ceramics treated with hydrochloric acid with a concentration of 0.5 mol/L, the number of immobilized E. coli cells is 1.2×10⁹ cells/cm³, and the number of immobilized magnetotactic bacterium cells is 1.1×10⁹ cells/cm³; on the macroporous ceramics treated with hydrochloric acid with a concentration of 1 mol/L, the number of immobilized E. coli cells is 1.4×10⁹ cells/cm³, and the number of immobilized magnetotactic bacterium cells is 1.3×10⁹ cells/cm³; on the macroporous ceramics treated with hydrochloric acid with a concentration of 1.5 mol/L, the number of immobilized E. coli cells is 1.3×10⁹ cells/cm³, and the number of immobilized magnetotactic bacterium cells is 1.3×10⁹ cells/cm³; on the macroporous ceramics treated with sodium hydroxide with a concentration of 0.5 mol/L, the number of immobilized E. coli cells is 1.3×10⁹ cells/cm³, and the number of immobilized magnetotactic bacterium cells is 1.2×10⁹ cells/cm³; on the macroporous ceramics treated with sodium hydroxide with a concentration of 1 mol/L, the number of immobilized E. coli cells is 1.3×10⁹ cells/cm³, and the number of immobilized magnetotactic bacterium cells is 1.3×10⁹ cells/cm³; and on the macroporous ceramics treated with sodium hydroxide with a concentration of 1.5 mol/L, the number of immobilized E. coli cells is 1.2×10⁹ cells/cm³, and the number of immobilized magnetotactic bacterium cells is 1.3×10⁹ cells/cm³.

Therefore, hydrochloric acid with a concentration of 1 mol/L shall be used to treat the macroporous ceramics to make the ceramics carry more positive charges or negative charges. Under such condition, the bacteria can better adhere to the gaps inside the ceramics by electrostatic adsorption, so that the inside of the ceramics is better filled, the metal adsorption rate is improved, and further the conductivity is higher.

### EXAMPLE 3-2

### Effect of Temperature on Immobilization of Microbes on Macroporous Ceramics (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 30° C., 40° C., 50° C. and 60° C. for 180 min, and dried at 105° C. for 12 h to obtain macroporous ceramics with immobilized E. coli and macroporous ceramics with immobilized magnetotactic bacteria.

The number of microbes immobilized on the macroporous ceramics was detected.

The detection results are: on the macroporous ceramics treated at 30° C., the number of immobilized E. coli cells is 1.1×10⁹ cells/cm³, and the number of immobilized magnetotactic bacterium cells is 1.2×10⁹ cells/cm³; on the macroporous ceramics treated at 40° C., the number of immobilized E. coli cells is 1.2×10⁹ cells/cm³, and the number of immobilized magnetotactic bacterium cells is 1.3×10⁹ cells/cm³; on the macroporous ceramics treated at 50° C., the number of immobilized E. coli cells is 1.3×10⁹ cells/cm³, and the number of immobilized magnetotactic bacterium cells is 1.4×10⁹ cells/cm³; and on the macroporous ceramics treated at 60° C., the number of immobilized E. coli cells is 1.3×10⁹ cells/cm³, and the number of immobilized magnetotactic bacterium cells is 1.3×10⁹ cells/cm³.

Therefore, the macroporous ceramics shall be treated at 50° C. to make the bacteria better adhere to the gaps inside the ceramics, so that the inside of the ceramics is better filled, the metal adsorption rate is improved, and further the conductivity is higher.

### EXAMPLE 3-3

### Effect of Rotation Speed on Immobilization of Microbes on Macroporous Ceramics (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution and the magnetotactic bacterium solution respectively, shaken on a shaker at 20 r·min⁻¹, 30 r·min⁻¹, 40 r·min⁻¹, 50 r·min⁻¹ and 60 r·min⁻¹ and 50° C. for 180 min, and dried at 105° C. for 12 h to obtain macroporous ceramics with immobilized E. coli and macroporous ceramics with immobilized magnetotactic bacteria.

The number of microbes immobilized on the macroporous ceramics was detected.

The detection results are: on the macroporous ceramics treated at 20 r·min⁻¹, the number of immobilized E. coli cells is 1.0×10⁹ cells/cm³, and the number of immobilized magnetotactic bacterium cells is 1.2×10⁹ cells/cm³; on the macroporous ceramics treated at 30 r·min⁻¹, the number of immobilized E. coli cells is 1.1×10⁹ cells/cm³, and the number of immobilized magnetotactic bacterium cells is 1.3×10⁹ cells/cm³; on the macroporous ceramics treated at 40 r·min⁻¹, the number of immobilized E. coli cells is 1.3×10⁹ cells/cm³, and the number of immobilized magnetotactic bacterium cells is 1.4×10⁹ cells/cm³; on the macroporous ceramics treated at 50 r·min⁻¹, the number of immobilized E. coli cells is 1.3×10⁹ cells/cm³, and the number of immobilized magnetotactic bacterium cells is 1.3×10⁹ cells/cm³; and on the macroporous ceramics treated at 60 r·min⁻¹, the number of immobilized E. coli cells is 1.3×10⁹ cells/cm³, and the number of immobilized magnetotactic bacterium cells is 1.2×10⁹ cells/cm³.

Therefore, the macroporous ceramics shall be treated at 40 r·min⁻¹ to make the bacteria better adhere to the gaps inside the ceramics without being thrown off, so that the inside of the ceramics is better filled, the metal adsorption rate is improved, and further the conductivity is higher.

### EXAMPLE 3-4

### Effect of Flow Rate on Adsorption of Metal Ions by Microbes (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 50° C. for 180 min, and dried at 105° C. for 12 h to obtain macroporous ceramics with immobilized E. coli and macroporous ceramics with immobilized magnetotactic bacteria.

(7) The macroporous ceramics with immobilized E. coli and the macroporous ceramics with immobilized magnetotactic bacteria were respectively fixed in soft catheters with two ends mutually communicated, and the catheters were connected to a peristaltic pump. The flow rate of the peristaltic pump was adjusted to 5 mL/min, 10 mL/min, 15 mL/min and 20 mL/min, and the two ends of the catheter were put into an ion solution with a molybdenum ion concentration of 65 mg/mL and a pH of 3. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics.

The amount of metal ions adsorbed by microbes is detected.

The detection results are: on the macroporous ceramics treated at a flow rate of 5 mL/min, the amount of metal ions adsorbed by E. coli is 1.1 mmol/g, and the amount of metal ions adsorbed by magnetotactic bacteria is 1.1 mmol/g; on the macroporous ceramics treated at a flow rate of 10 mL/min, the amount of metal ions adsorbed by E. coli is 1.3 mmol/g, and the amount of metal ions adsorbed by magnetotactic bacteria is 1.3 mmol/g; on the macroporous ceramics treated at a flow rate of 15 mL/min, the amount of metal ions adsorbed by E. coli is 1.2 mmol/g, and the amount of metal ions adsorbed by magnetotactic bacteria is 1.3 mmol/g; and on the macroporous ceramics treated at a flow rate of 20 mL/min, the amount of metal ions adsorbed by E. coli is 1.2 mmol/g, and the amount of metal ions adsorbed by magnetotactic bacteria is 1.2 mmol/g.

Therefore, the macroporous ceramics shall be treated at a flow rate of 10 mL/min.

### EXAMPLE 3-5

### Effect of pH on Adsorption of Metal Ions by Microbes (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 50° C. for 180 min, and dried at 105° C. for 12 h to obtain macroporous ceramics with immobilized E. coli and macroporous ceramics with immobilized magnetotactic bacteria.

(7) The macroporous ceramics with immobilized E. coli and the macroporous ceramics with immobilized magnetotactic bacteria were respectively fixed in soft catheters with two ends mutually communicated, and the catheters were connected to a peristaltic pump. The flow rate of the peristaltic pump was adjusted to 10 mL/min, and the two ends of the catheter were put into ion solutions with a molybdenum ion concentration of 65 mg/mL and a pH of 1, 2, 3, 4 and 5 respectively. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 105° C. for 12 h to obtain microbial conductive ceramics.

The amount of metal ions adsorbed by microbes is detected.

The detection results are: on the macroporous ceramics treated at a pH of 1, the amount of metal ions adsorbed by E. coli is 1.1 mmol/g, and the amount of metal ions adsorbed by magnetotactic bacteria is 1.2 mmol/g; on the macroporous ceramics treated at a pH of 2, the amount of metal ions adsorbed by E. coli is 1.2 mmol/g, and the amount of metal ions adsorbed by magnetotactic bacteria is 1.2 mmol/g; on the macroporous ceramics treated at a pH of 3, the amount of metal ions adsorbed by E. coli is 1.3 mmol/g, and the amount of metal ions adsorbed by magnetotactic bacteria is 1.3 mmol/g; on the macroporous ceramics treated at a pH of 4, the amount of metal ions adsorbed by E. coli is 1.3 mmol/g, and the amount of metal ions adsorbed by magnetotactic bacteria is 1.4 mmol/g; and on the macroporous ceramics treated at a pH of 5, the amount of metal ions adsorbed by E. coli is 1.3 mmol/g, and the amount of metal ions adsorbed by magnetotactic bacteria is 1.3 mmol/g.

Therefore, the macroporous ceramics shall be treated at a pH of 4.

### EXAMPLE 3-6

### Effect of Time on Adsorption of Metal Ions by Microbes (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 48 h, 60 h, 72 h, 84 h and 96 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 50° C. for 180 min, and dried at 105° C. for 12 h to obtain macroporous ceramics with immobilized E. coli and macroporous ceramics with immobilized magnetotactic bacteria.

(7) The macroporous ceramics with immobilized E. coli and the macroporous ceramics with immobilized magnetotactic bacteria were respectively fixed in soft catheters with two ends mutually communicated, and the catheters were connected to a peristaltic pump. The flow rate of the peristaltic pump was adjusted to 10 mL/min, and the two ends of the catheter were put into an ion solution with a molybdenum ion concentration of 65 mg/mL and a pH of 4. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 150° C. for 2 h to obtain microbial conductive ceramics.

The amount of metal ions adsorbed by microbes is detected.

The detection results are: after fermentation culture for 48 h, the amount of metal ions adsorbed by E. coli is 1.1 mmol/g, and the amount of metal ions adsorbed by magnetotactic bacteria is 1.1 mmol/g; after fermentation culture for 60 h, the amount of metal ions adsorbed by E. coli is 1.2 mmol/g, and the amount of metal ions adsorbed by magnetotactic bacteria is 1.3 mmol/g; after fermentation culture for 72 h, the amount of metal ions adsorbed by E. coli is 1.3 mmol/g, and the amount of metal ions adsorbed by magnetotactic bacteria is 1.4 mmol/g; after fermentation culture for 84 h, the amount of metal ions adsorbed by E. coli is 1.3 mmol/g, and the amount of metal ions adsorbed by magnetotactic bacteria is 1.4 mmol/g; and after fermentation culture for 96 h, the amount of metal ions adsorbed by E. coli is 1.3 mmol/g, and the amount of metal ions adsorbed by magnetotactic bacteria is 1.3 mmol/g.

Therefore, treatment with the microbial macroporous ceramics subjected to fermentation culture for 48-96 h has good effects. It may be because the bacteria are in the logarithmic growth phase, the stable phase or the transition phase from the logarithmic growth phase to the stable phase, the cell membrane has better permeability, and the metal ions can be absorbed more easily.

### EXAMPLE 3-7

### Preparation of Microbial Conductive Ceramics (Bacteria)

Specific steps are as follows:

(1) A single colony of E. coli was picked from a plate, inoculated into a 500 mL Erlenmeyer flask pre-added with 50 mL of seed medium, and cultured in a shaker at 37° C. and 220 r·min⁻¹ for 60 h to obtain an E. coli seed solution.

(2) The E. coli seed solution was inoculated into a 5 L fermenter pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution was centrifuged at 1500 r·min⁻¹ for 15 min to obtain bacterial cells; the bacterial cells were washed with distilled water and centrifuged at 1500 r·min⁻¹ for 5 min, and live bacterial cells were collected; and the washing operation was repeated 3 times to obtain E. coli cells.

(4) The E. coli cells were put into distilled water, and the cell concentration was controlled at 1×10⁹ cells/mL to obtain an E. coli solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution, shaken on a shaker at 40 r·min⁻¹ and 50° C. for 180 min, and dried at 105° C. for 12 h to obtain macroporous ceramics with immobilized E. coli.

(7) The macroporous ceramics with immobilized E. coli were fixed in a soft catheter with two ends mutually communicated, and the catheter was connected to a peristaltic pump. The flow rate of the peristaltic pump was adjusted to 10 mL/min, and the two ends of the catheter were put into an ion solution with a molybdenum ion concentration of 65 mg/mL and a pH of 4. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 105° C. for 12 h to obtain microbial conductive ceramics. The conductive properties of the microbial conductive ceramics were detected, and the conductivity was 2.41×10⁶ S/m.

### EXAMPLE 3-8

### Preparation of Microbial Conductive Ceramics (Bacteria)

Specific steps are as follows:

(1) A single colony of magnetotactic bacteria was picked from a plate, inoculated into a 500 mL Erlenmeyer flask pre-added with 50 mL of seed medium, and cultured in a shaker at 37° C. and 220 r·min⁻¹ for 60 h to obtain a magnetotactic bacterium seed solution.

(2) The magnetotactic bacterium seed solution was inoculated into a 5 L fermenter pre-added with 1.2 L of fermentation medium at an inoculum amount of 10% (that is, the volume of the seed solution accounted for 10% of the volume of the fermentation medium), and fermented at 37° C. for 72 h to obtain a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained magnetotactic bacterium fermentation solution was centrifuged at 1500 r·min⁻¹ for 15 min to obtain bacterial cells; the bacterial cells were washed with distilled water and centrifuged at 1500 r·min⁻¹ for 5 min, and live bacterial cells were collected; and the washing operation was repeated 3 times to obtain magnetotactic bacterium cells.

(4) The magnetotactic bacterium cells were put into distilled water, and the cell concentration was controlled at 1×10⁹ cells/mL to obtain a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the magnetotactic bacterium solution, shaken on a shaker at 40 r·min⁻¹ and 50° C. for 180 min, and dried at 105° C. for 12 h to obtain macroporous ceramics with immobilized magnetotactic bacteria.

(7) The macroporous ceramics with immobilized magnetotactic bacteria were fixed in a soft catheter with two ends mutually communicated, and the catheter was connected to a peristaltic pump. The flow rate of the peristaltic pump was adjusted to 10 mL/min, and the two ends of the catheter were put into an ion solution with a molybdenum ion concentration of 65 mg/mL and a pH of 4. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 105° C. for 12 h to obtain microbial conductive ceramics. The conductive properties of the microbial conductive ceramics were detected, and the conductivity was 2.40×10⁶ S/m.

### EXAMPLE 3-9

### Preparation of Microbial Conductive Ceramics (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 50° C. for 180 min, and dried at 105° C. for 12 h to obtain macroporous ceramics with immobilized E. coli and macroporous ceramics with immobilized magnetotactic bacteria.

(7) The macroporous ceramics with immobilized E. coli and the macroporous ceramics with immobilized magnetotactic bacteria were fixed in soft catheters with two ends mutually communicated, and the catheters were connected to a peristaltic pump. The flow rate of the peristaltic pump was adjusted to 10 mL/min, and the two ends of the catheter were put into an ion solution with a molybdenum ion concentration of 65 mg/mL and a pH of 4. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 105° C. for 12 h to obtain microbial conductive ceramics. The conductive properties of the microbial conductive ceramics were detected, and the conductivity was 2.41×10⁶ S/m.

### EXAMPLE 3-10

### Preparation of Microbial Conductive Ceramics (Bacteria)

Specific steps are as follows:

(1) A single colony of E. coli and a single colony of magnetotactic bacteria were picked from plates, inoculated into 500 mL Erlenmeyer flasks pre-added with 50 mL of seed medium respectively, and cultured in shakers at 37° C. and 220 r·min⁻¹ for 60 h to obtain an E. coli seed solution and a magnetotactic bacterium seed solution.

(2) The E. coli seed solution and the magnetotactic bacterium seed solution were respectively inoculated into 5 L fermenters pre-added with 1.2 L of fermentation medium at an inoculum amount of 10% (that is, the volume of the seed solution accounted for 10% of the volume of the fermentation medium), and fermented at 37° C. for 72 h to obtain an E. coli fermentation solution and a magnetotactic bacterium fermentation solution; in the whole fermentation process, the aeration volume and stirring speed need to be adjusted to control the dissolved oxygen not to be less than 10% in the fermentation solution, and glucose and peptone need to be fed to control the glucose content to be not less than 60 g/L and the peptone content to be not less than 15 g/L in the fermentation solution (to supplement the carbon source and nitrogen source consumed in the cell growth process).

(3) The obtained E. coli fermentation solution and magnetotactic bacterium fermentation solution were centrifuged at 1500 r·min⁻¹ for 15 min respectively to obtain bacterial cells; the bacterial cells were washed with distilled water and centrifuged at 1500 r·min⁻¹ for 5 min, and live bacterial cells were collected; and the washing operation was repeated 3 times to obtain E. coli cells and magnetotactic bacterium cells.

(4) The E. coli cells and the magnetotactic bacterium cells were put into distilled water respectively, and the cell concentration was controlled at 1×10⁹ cells/mL to obtain an E. coli solution and a magnetotactic bacterium solution.

(5) Macroporous ceramics were soaked in hydrochloric acid with a concentration of 1 mol/L for 24 h, and then dried at 105° C. for 12 h to obtain treated macroporous ceramics.

(6) The treated macroporous ceramics were put into the E. coli solution and the magnetotactic bacterium solution respectively, shaken on a shaker at 40 r·min⁻¹ and 50° C. for 180 min, and dried at 105° C. for 12 h to obtain macroporous ceramics with immobilized E. coli and macroporous ceramics with immobilized magnetotactic bacteria.

(7) The macroporous ceramics with immobilized E. coli and the macroporous ceramics with immobilized magnetotactic bacteria were fixed in soft catheters with two ends mutually communicated, and the catheters were connected to a peristaltic pump. The flow rate of the peristaltic pump was adjusted to 10 mL/min, and the two ends of the catheter were put into ion solutions with a concentration of 65 mg/mL of silver ions, copper ions and aluminum ions respectively and with a pH of 4. The peristaltic pump was turned on at 40° C., and the metal ion concentrate was fed to the soft catheter. The concentrate slowly passed through the ceramics, and then the metal ions were adsorbed for 90 min. After the adsorption, the macroporous ceramics with immobilized microbes were dried at 105° C. for 12 h to obtain microbial conductive ceramics. The conductive properties of the microbial conductive ceramics were detected, the experiment was repeated three times, and the conductivity was 2.51×10⁶ S/m, 2.31×10⁶ S/m and 2.24×10⁶ S/m respectively.

What is claimed is:

1. A preparation method of microbial conductive ceramics, comprising: culturing microbes in a culture medium to a logarithmic growth phase or a stable phase to obtain a microbial bacterial solution; soaking macroporous ceramics in a hydrochloric acid or sodium hydroxide solution and then drying the macroporous ceramics for the first time to obtain pretreated macroporous ceramics; placing the pretreated macroporous ceramics into the microbial bacterial solution for shaking and then drying the macroporous ceramics for the second time to obtain macroporous ceramics with immobilized microbes; and passing a metal ion solution through the macroporous ceramics with immobilized microbes, and then drying the macroporous ceramics for the third time to obtain the microbial conductive ceramics, wherein the microbes comprise saccharomycetes, filamentous fungi or bacteria.

2. The preparation method according to claim 1, wherein when the microbes are saccharomycetes, the culture time of the microbes in the culture medium is 12-60 h; when the microbes are filamentous fungi, the culture time of the microbes in the culture medium is 24-72 h; and when the microbes are bacteria, the culture time of the microbes in the culture medium is 48-96 h.

3. The preparation method according to claim 1, wherein when the microbes are saccharomycetes, a cell concentration of the microbial bacterial solution is 1×10⁶-1×10¹⁰ cells/mL; when the microbes are filamentous fungi, a cell concentration of the microbial bacterial solution is 1×10⁶-1×10⁸ cells/mL; and when the microbes are bacteria, a cell concentration of the microbial bacterial solution is 1×10⁸-1×10¹⁰ cells/mL.

4. The preparation method according to claim 1, wherein when the microbes are saccharomycetes, a pore size of macroporous ceramics is 10-20 μm; when the microbes are filamentous fungi, a pore size of macroporous ceramics is 50-200 μm; and when the microbes are bacteria, a pore size of macroporous ceramics is 1-10 μm.

5. The preparation method according to claim 1, wherein when the microbes are saccharomycetes, filamentous fungi or bacteria, a concentration of the hydrochloric acid is 0.5-1.5 mol/L.

6. The preparation method according to claim 1, wherein when the microbes are saccharomycetes, filamentous fungi or bacteria, a concentration of the sodium hydroxide is 0.5-1.5 mol/L.

7. The preparation method according to claim 1, wherein when the microbes are saccharomycetes, filamentous fungi or bacteria, the soaking is performed at a temperature of 20-30° C. for 24-48 h.

8. The preparation method according to claim 1, wherein when the microbes are saccharomycetes, the shaking is performed at a rotation speed of 50-100 r/min and at a temperature of 30-50° C. for 60-150 min; when the microbes are filamentous fungi, the shaking is performed at a rotation speed of 120-200 r/min and at a temperature of 20-40° C. for 4-8 h; and when the microbes are bacteria, the shaking is performed at a rotation speed of 20-60 r/min and at a temperature of 40-60° C. for 120-240 min.

9. The preparation method according to claim 1, wherein when the microbes are saccharomycetes or filamentous fungi, a concentration of the metal ion solution is 30-100 mg/mL; and when the microbes are bacteria, a concentration of the metal ion solution is 50-80 mg/mL.

10. The preparation method according to claim 1, wherein when the microbes are saccharomycetes, filamentous fungi or bacteria, a pH of the metal ion solution is 2-5.

11. The preparation method according to claim 1, wherein when the microbes are saccharomycetes, the passing the metal ion solution through the macroporous ceramics with immobilized microbes is performed at a temperature of 15-35° C. and at a flow rate of 10-30 mL/min for 30-120 min; when the microbes are filamentous fungi, the passing the metal ion solution through the macroporous ceramics with immobilized microbes is performed at a temperature of 45-55° C. and at a flow rate of 20-40 mL/min for 150-240 min; and when the microbes are bacteria, the passing the metal ion solution through the macroporous ceramics with immobilized microbes is performed at a temperature of 35-45° C. and at a flow rate of 5-20 mL/min for 60-150 min.

12. Microbial conductive ceramics prepared by the method according to claim 1.

13. Microbial conductive ceramics, comprising macroporous ceramics, microbes immobilized on the macroporous ceramics and metal ions adsorbed to the microbes, wherein the microbes comprise saccharomycetes, filamentous fungi or bacteria.

14. The microbial conductive ceramics according to claim 13, wherein the saccharomycetes comprises Saccharomyces cerevisiae and/or Pichia pastoris; the filamentous fungi comprise one or more of Aspergillus niger, Aspergillus oryzae or Mucor; and the bacteria comprise Escherichia coli and/or magnetotactic bacteria.

15. The microbial conductive ceramics according to claim 13, wherein when the microbes are saccharomycetes, filamentous fungi or bacteria, the macroporous ceramics comprise one or more of silicon nitride ceramics, alumina ceramics, zirconia ceramics or titanium aluminum carbide ceramics.

16. The microbial conductive ceramics according to claim 13, wherein when the microbes are saccharomycetes, a pore size of macroporous ceramics is 10-20 μm; when the microbes are filamentous fungi, a pore size of macroporous ceramics is 50-200 μm; and when the microbes are bacteria, a pore size of macroporous ceramics is 1-10 μm.

17. The microbial conductive ceramics according to claim 13, wherein when the microbes are saccharomycetes, filamentous fungi or bacteria, the metal ions comprise one or more of silver ion, molybdenum ion, aluminum ion, or copper ion.

18. A product comprising the microbial conductive ceramics according to claim 12.

19. The product according to claim 18, comprising an electronic component, an electric heating element, an electrode, a battery, an electronic camera, a television, a radio, a computer or a mobile television.
